(19) United States
(12) Patent Application Publication
(10) Pub. No.: US 2013/0030054 A1
(43) Pub. Date: Jan. 31, 2013

(54) HIGHLY FLAVORED ORNITHINE-CONTAINING ALCOHOL-FREE MALT BEVERAGE

(71) Applicants: Yukimasa Owa, Chuo-ku (JP); Takehito Ota, Chuo-ku (JP); Kinya Kurihara, Chuo-ku (JP)

(72) Inventors: Yukimasa Owa, Chuo-ku (JP); Takehito Ota, Chuo-ku (JP); Kinya Kurihara, Chuo-ku (JP)

(73) Assignees: KIRIN HOLDINGS KABUSHIKI KAISHA, Chuo-ku, Tokyo (JP); KIRIN BEER KABUSHIKI KAISHA, Chuo-ku, Tokyo (JP); KYOWA HAKKO BIO CO., LTD., Chiyoda-ku, Tokyo (JP); KOIWAI DAIRY PRODUCTS CO., LTD., Chiyoda-ku, Tokyo (JP); KIRIN BEVERAGE COMPANY, LIMITED, Chiyoda-ku, Tokyo (JP)

(21) Appl. No.: 13/520,724

(22) PCT Filed: Dec. 28, 2010
(86) PCT No.: PCT/JP2010/007584

§ 371 Date: Oct. 1, 2012

(30) Foreign Application Priority Data
Jan. 8, 2010 (JP) 2010-003191

(51) Int. Cl. A61K 31/198

(52) U.S. Cl. 514/564

(57) ABSTRACT

An object of the present invention is to provide an ornithine-containing alcohol-free malt beverage which is provided with health functions of ornithine imparted to an alcoholic beverage-flavored alcohol-free malt beverage containing substantially no alcohol component and which retains an excellent flavor of the alcohol-free malt beverage, and to provide a method for producing the same. The ornithine-containing alcohol-free malt beverage of the present invention is produced using ornithine hydrochloride and ornithine aspartate in combination as ornithine raw materials. For this production, 20% or more by weight of ornithine hydrochloride in terms of ornithine is used with respect to the total weight of ornithine hydrochloride and ornithine aspartate in terms of ornithine. The ornithine-containing alcohol-free malt beverage thus produced is provided with health functions of ornithine and retains an excellent flavor of an alcohol-free malt beverage. In this way, the object can be attained. In the present invention, an organic acid, in addition to ornithine hydrochloride and ornithine aspartate, may be added to adjust the pH value of the product. Examples of the added organic acid include one or more organic acids selected from malic acid, gluconic acid and phytic acid.

### TECHNICAL FIELD

The present invention relates to an ornithine-containing alcohol-free malt beverage which is provided with health functions of ornithine and retains an excellent flavor of an alcohol-free malt beverage, and to a method for producing the same.

### BACKGROUND ART

Alcoholic beverage-flavored alcohol-free fermented beverages, which are derived from alcoholic beverages and have an alcohol content less than 1%, have been developed in recent years as beverages have grown more diverse. The alcohol-free fermented beverages having an alcohol content less than 1% are produced by various known methods, for example, a method involving minimizing the fermentation of fermentable sugars in the production process, a method involving preventing alcohol formation by use of specific yeast, a method involving adjusting an alcohol component to less than 1% by evaporation after production of alcoholic beverages, a method involving adjusting an alcohol component to less than 1% by dialysis after production of alcoholic beverages, and a method involving treating alcoholic beverages with an asymmetric reverse osmosis membrane to adjust an alcohol component to less than 1%.

Recently, there has also been disclosed a method for producing an alcohol-free fermented beverage such as alcohol-free beer, comprising adding a-glucosidase in a wort production step during beer production to convert fermentable sugars in a saccharified solution to non-fermentable sugars so that the concentration of alcohols formed in a fermentation step is lowered in fermented beverage production (Japanese unexamined Patent Application Publication No. 5-68528). The alcohol component in all of such disclosed alcohol-free fermented beverages is adjusted to less than 1%. In any case, however, fermentation is performed in the production process thereof, and thus, the production fails to give alcoholic beverage-flavored alcohol-free beverages containing substantially no alcohol component.

With people becoming increasingly concerned about the effects of foods and drinks on health, healthy foods and drinks have been developed actively. Various components having health functions for this purpose have been disclosed. Ornithine is known as such a component having health functions. Ornithine has heretofore been known to have various health functions, for example, various physiological functions such as anti-fatigue effect (WO2004/078171), blood-alcohol level-lowering effect (WO2007/023931), sleep-improving effect (Japanese unexamined Patent Application Publication No. 2006-342148), blood flow-improving effect (WO2007/049628) and muscle mass-increasing effect (WO2007/077995). This component has been utilized in healthy foods or the like by addition to various foods and drinks or, usually, by tableting.

Since ornithine itself is difficult to use due to its alkalinity, it is sold in the form of ornithine hydrochloride as raw materials for foods and drinks. Ornithine hydrochloride, however, disadvantageously increases chloride ion concentrations in an aqueous solution. Also unfortunately, ornithine hydrochloride having a concentration exceeding a certain level has an adverse influence, such as corrosion, on production equipment (e.g., tanks and piping) or can containers, or gives perceivable salty taste when added to dairy products. For these reasons, ornithine aspartate is used instead. Ornithine aspartate is free from problems associated with its use as in ornithine hydrochloride, owing to the absence of chloride ions. Unfortunately, off-taste unique to ornithine aspartate reduces the palatability of produced ornithine aspartate-containing dairy products.

Thus, there has also been disclosed methods for reducing the off-taste of amino acids (e.g., ornithine) or peptides. For example, Japanese Patent Publication No. 3-47829 discloses a food composition which is allowed to contain amino acids such as isoleucine, leucine, lysine, or ornithine and sugars under conditions free from browning reaction and which is provided with a cacao or coffee flavor to reduce a feeling of resistance toward bitterness. Japanese unexamined Patent Application Publication No. 2009-118743 discloses a method for preventing the bitterness of amino acids, comprising adding a thickener such as xanthan gum or guar gum and poly-γ-glutamic acid.

As described above, with people becoming increasingly concerned about the effects of foods and drinks on health, attempts have been made to add ornithine to foods and drinks to impart health functions of ornithine to the foods and drinks. Also, there have been disclosed methods for reducing the off-taste of amino acids (e.g., ornithine) or peptides generated in such a case. None of the previous approaches, however, have proposed an attempt to impart health functions of ornithine or the like to alcohol-free malt beverages or beverages in which problems that add constraints in such a case have been solved.

### PRIOR ART DOCUMENTS

### Patent Documents

Patent Document 1: Japanese Patent Publication No. 3-47829
Patent Document 2: Japanese unexamined Patent Application Publication No. 5-68528
Patent Document 3: Japanese unexamined Patent Application Publication No. 2006-342148
Patent Document 4: Japanese unexamined Patent Application Publication No. 2009-118743
Patent Document 5: WO2004/078171
Patent Document 6: WO2007/023931
Patent Document 7: WO2007/049628
Patent Document 8: WO2007/077995

### SUMMARY OF THE INVENTION

### Object to be Solved by the Invention

An object of the present invention is to provide an ornithine-containing alcohol-free malt beverage which is provided with health functions of ornithine imparted to an alcoholic beverage-flavored alcohol-free malt beverage containing substantially no alcohol component and which retains an excellent flavor of the alcohol-free malt beverage, and to provide a method for producing the same. Particularly, an object of the present invention is to provide an ornithine-containing alcohol-free malt beverage which is provided with health functions of ornithine imparted to an alcohol-free malt beverage and which has sourness balanced with umami as a beer-like beverage, a beer-like flavor, and reduced taste derived from a pH adjuster or ornithine, and to provide a method for producing the same.

### Means to Solve the Object

When health functions are imparted to alcohol-free malt beverages by the addition of ornithine, the added ornithine material increases the pH value or buffering capacity of wort. This requires excessive sterilization conditions based on the food sanitation law. Alternatively, pH adjustment for circumventing this problem disadvantageously increases the intensity of sourness and deletes taste as beverages. Depending on type, the added ornithine material also imparts excessive umami or its off-taste to beverages and presents problems associated with the taste of beverages.

Thus, the present inventors have conducted diligent studies to solve such problems associated with the addition of ornithine to alcohol-free malt beverages and to produce an ornithine-containing alcohol-free malt beverage which is provided with health functions of ornithine imparted to an alcohol-free malt beverage and which retains an excellent flavor of the alcohol-free malt beverage. Consequently, the present inventors have found that: increase in the pH value or buffering capacity of wort caused by the addition of ornithine or off-taste imparted by the addition of the ornithine material can be circumvented by using ornithine hydrochloride and ornithine aspartate as ornithine raw materials and adding them at a particular weight ratio to a beverage; and the ornithine-containing alcohol-free malt beverage thus produced is provided with health functions of ornithine and retains an excellent flavor of an alcohol-free malt beverage. Based on these findings, the present invention has been completed.

The present invention is directed to the production of an ornithine-containing alcohol-free malt beverage which is provided with health functions of ornithine and retains an excellent flavor of an alcohol-free malt beverage, by using ornithine hydrochloride and ornithine aspartate in combination as ornithine raw materials to produce the ornithine-containing alcohol-free malt beverage, in which 20% or more by weight of ornithine hydrochloride in terms of ornithine is used with respect to the total weight of ornithine hydrochloride and ornithine aspartate in terms of ornithine. In the present invention, the weight ratio between the amounts of ornithine hydrochloride and ornithine hydrochloride used is indicated on the basis of a weight in terms of ornithine.

Specifically, in an attempt to impart health functions of ornithine to an alcohol-free malt beverage, the major problem of the added ornithine material is to increase the pH value or buffering capacity of wort. In addition, ornithine aspartate, for example, contains 50% aspartic acid and as such, disadvantageously causes excessive umami components leading to unpleasant aftertaste due to the aspartic acid concentration increased along with ornithine concentration. Briefly, the following two problems arise: (1) the increased buffering capacity of wort; and (2) excessive umami components leading to unpleasant aftertaste. Thus, (1) the increased buffering capacity of wort is specifically disadvantageous in the following: for alcohol-free beverages, which are included in beverages based on the food sanitation law (Notification No. 213, Ministry of Health, Labour and Welfare, Japan), it is required that wort of pH less than 4.0 should be sterilized at 65° C.×10 minutes or under equivalent or more strict conditions and wort of pH 4.0 to 4.6 should be sterilized at 85° C.×30 minutes or under equivalent or more strict conditions. Particularly, when a pH value less than 4.0 is selected for a product in its design, it is required to greatly lower the pH value of the product using an organic acid or the like under constraints of sterilization equipment or from the viewpoint of energy cost or the like required for sterilization. In this case, the problem is strong sourness.

Such pH adjustment brings about strong sourness even in usual alcohol-free malt beverages, as described above, and further increases the intensity of sourness in products having a pH value increased due to the ornithine material used. The products thus prepared are very hardly drinkable. In this regard, ornithine hydrochloride, which is an ornithine material that does not increase pH, may be used. This can reduce the amount of the organic acid used and, however, increases the risk of corroding production equipment, can materials for final products, or the like, due to the increased concentration of chloride ions derived from ornithine hydrochloride. For a solution, ornithine hydrochloride can be used in combination with an additional ornithine material to lower the concentration of chloride ions derived from ornithine hydrochloride.

Moreover, the present inventors have contemplated coping with the excessive umami components leading to unpleasant aftertaste by the following solution: for example, use of ornithine aspartate strengthens aspartic acid-derived umami leading to unpleasant aftertaste, depending on the amount of ornithine aspartate used. Thus, ornithine hydrochloride is free from such a substance leading to umami and can therefore be used at a percentage increased to some extent to produce effects including the reduction of excessive umami.

As described above, the present inventors have found that use of ornithine hydrochloride and ornithine aspartate in combination can lower the concentration of chloride ions derived from ornithine hydrochloride and also achieve the preparation of the optimum wort without increasing the pH value of wort. Furthermore, an ornithine material containing an amino acid other than ornithine, such as ornithine aspartate, can be used in combination with an increased percentage of ornithine hydrochloride to reduce excessive umami. In this way, the present inventors have successfully provided an ornithine-containing alcohol-free malt beverage excellent in flavor.

In the present invention, it is preferred that ornithine hydrochloride and ornithine aspartate should be contained in an amount of at least 0.05% or more by weight in terms of ornithine per product of the alcohol-free malt beverage. In the present invention, it is also preferred that ornithine aspartate should be contained in an amount of 500 ppm or lower.

For the production of the ornithine-containing alcohol-free malt beverage of the present invention, an organic acid may be added to adjust the pH value of the product, in addition to using ornithine hydrochloride and ornithine aspartate in combination as ornithine raw materials to produce the ornithine-containing alcohol-free malt beverage. Examples of the added organic acid include one or more organic acids selected from malic acid, gluconic acid and phytic acid. It is preferred that the organic acid should be added to adjust the pH value of the product to less than 3.80. In this addition, the organic acids described above may be used in combination with an additional organic acid, for example, lactic acid.

Regarding the relationship of the amount of the organic acid added with the weight ratio between the amounts of ornithine hydrochloride and ornithine aspartate used, for example, when the amount of the malic acid added is 0.058 to 0.078% v/v with respect to wort, 80% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=80:20.

Furthermore, when the amount of the gluconic acid added is 0.145 to 0.24% v/v with respect to wort, 60% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=60:40 to 80:20, more preferably 60:40. Alternatively, when the amount of the phytic acid added is 0.03 to 0.06% v/v with respect to wort, 20% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=20:80 to 80:20, more preferably 20:80 to 60:40.

The alcohol-free malt beverage according to the present invention is a malt beverage containing substantially no alcohol. Examples of the alcohol-free malt beverage can include alcohol-free malt beverages that are produced by a nonfermentation process of wort involving a saccharification step, a wort boiling step, a low-temperature treatment step for the boiled wort, and a wort flavor removal step by adsorption treatment and produced without undergoing a fermentation step in the production of the alcohol-free malt beverages.

The present invention encompasses an ornithine-containing alcohol-free malt beverage which is provided with health functions of ornithine and retains an excellent flavor of an alcohol-free malt beverage, the ornithine-containing alcohol-free malt beverage being produced by the method of the present invention for producing an alcohol-free malt beverage.

Specifically; the present invention provides: (1) a method for producing an ornithine-containing alcohol-free malt beverage which is provided with health functions of ornithine and retains an excellent flavor of an alcohol-free malt beverage, comprising using ornithine hydrochloride and ornithine aspartate in combination as ornithine raw materials to produce the ornithine-containing alcohol-free malt beverage, wherein 20% or more by weight of ornithine hydrochloride in terms of ornithine is used with respect to the total weight of ornithine hydrochloride and ornithine aspartate in terms of ornithine; (2) the method for producing an ornithine-containing alcohol-free malt beverage which is provided with health functions of ornithine and retains an excellent flavor of an alcohol-free malt beverage according to (1), wherein 60% or more by weight of ornithine hydrochloride in terms of ornithine is used with respect to the total weight of ornithine hydrochloride and ornithine aspartate in terms of ornithine; (3) the method for producing an ornithine-containing alcohol-free malt beverage which is provided with health functions of ornithine and retains an excellent flavor of an alcohol-free malt beverage according to (2), wherein 80% or more by weight of ornithine hydrochloride in terms of ornithine is used with respect to the total weight of ornithine hydrochloride and ornithine aspartate in terms of ornithine; (4) the method for producing an ornithine-containing alcohol-free malt beverage which is provided with health functions of ornithine and retains an excellent flavor of an alcohol-free malt beverage according to any one of (1) to (3), wherein ornithine hydrochloride and ornithine aspartate are contained in an amount of at least 0.05% or more by weight in terms of ornithine per product of the alcohol-free malt beverage; and (5) the method for producing an ornithine-containing alcohol-free malt beverage which is provided with health functions of ornithine and retains an excellent flavor of an alcohol-free malt beverage according to any one of (1) to (4), wherein ornithine aspartate is contained in an amount of 500 ppm or lower.

The present invention also provides: (6) the method for producing an ornithine-containing alcohol-free malt beverage which is provided with health functions of ornithine and retains an excellent flavor of an alcohol-free malt beverage according to any one of (1) to (5), wherein the method further comprises adding an organic acid to adjust the pH value of the product, in addition to using ornithine hydrochloride and ornithine aspartate in combination as ornithine raw materials, to produce the ornithine-containing alcohol-free malt beverage; (7) the method for producing an ornithine-containing alcohol-free malt beverage which is provided with health functions of ornithine and retains an excellent flavor of an alcohol-free malt beverage according to (6), wherein the added organic acid is one or more organic acids selected from malic acid, gluconic acid and phytic acid; (8) the method for producing an ornithine-containing alcohol-free malt beverage which is provided with health functions of ornithine and retains an excellent flavor of an alcohol-free malt beverage according to (6) or (7), wherein the organic acid is added to adjust the pH value of the product to less than 3.80; (9) the method for producing an ornithine-containing alcohol-free malt beverage which is provided with health functions of ornithine and retains an excellent flavor of an alcohol-free malt beverage according to (6) or (7), wherein malic acid is used in an amount of 0.058 to 0.078% v/v with respect to wort, and 20% or more by weight of ornithine hydrochloride in terms of ornithine is used with respect to the total weight of ornithine hydrochloride and ornithine aspartate in terms of ornithine; and (10) the method for producing an ornithine-containing alcohol-free malt beverage which is provided with health functions of ornithine and retains an excellent flavor of an alcohol-free malt beverage according to (6) or (7), wherein gluconic acid is used in an amount of 0.145 to 0.24% v/v with respect to wort, and 40% or more by weight of ornithine hydrochloride in terms of ornithine is used with respect to the total weight of ornithine hydrochloride and ornithine aspartate in terms of ornithine.

The present invention further provides: (11) the method for producing an ornithine-containing alcohol-free malt beverage which is provided with health functions of ornithine and retains an excellent flavor of an alcohol-free malt beverage according to (6) or (7), wherein phytic acid is used in an amount of 0.03 to 0.06% v/v with respect to wort, and 80% or more by weight of ornithine hydrochloride in terms of ornithine is used with respect to the total weight of ornithine hydrochloride and ornithine aspartate in terms of ornithine; (12) the method for producing an ornithine-containing alcohol-free malt beverage which is provided with health functions of ornithine and retains an excellent flavor of an alcohol-free malt beverage according to any one of (1) to (11), wherein the alcohol-free malt beverage is produced by a nonfermentation process of wort; (13) an ornithine-containing alcohol-free malt beverage which is provided with health functions of ornithine and retains an excellent flavor of an alcohol-free malt beverage, produced by a method for producing an alcohol-free malt beverage according to any one of (1) to (12); and (14) an ornithine-containing alcohol-free malt beverage which is provided with health functions of ornithine and retains an excellent flavor of an alcohol-free malt beverage, comprising 0.05% or more by weight and 0.2% or less by weight of ornithine.

### Effect of the Invention

The present invention provides an ornithine-containing alcohol-free malt beverage which is provided with health functions of ornithine imparted to an alcoholic beverage-flavored alcohol-free malt beverage containing substantially no alcohol component and which retains an excellent flavor of the alcohol-free malt beverage.

### MODE OF CARRYING OUT THE INVENTION

The present invention provides a method for producing an ornithine-containing alcohol-free malt beverage which is provided with health functions of ornithine and retains an excellent flavor of an alcohol-free malt beverage, comprising using ornithine hydrochloride and ornithine aspartate in combination as ornithine raw materials to produce the ornithine-containing alcohol-free malt beverage, wherein 20% or more by weight of ornithine hydrochloride in terms of ornithine is used with respect to the total weight of ornithine hydrochloride and ornithine aspartate in terms of ornithine.

In the present invention, the alcohol-free malt beverage to which health functions of ornithine is imparted is prepared as a malt beverage containing substantially no alcohol by a preparation process free from a fermentation step. The production of the alcohol-free malt beverage includes, for example, a nonfermentation process of wort involving a wort preparation step, a wort boiling step, a low-temperature treatment step for the boiled wort, and a wort flavor removal step by adsorption treatment. Hereinafter, the production of the alcohol-free malt beverage will be described.

### (Preparation of Wort)

First, wort is prepared. The wort can be obtained according to a routine method by a process involving the steps of, for example, (a) saccharifying and filtering a mixture of a malt powder, secondary raw materials and water to obtain wort having a sugar content of 4 to 14%, (b) adding hops to the obtained wort, followed by boiling, and (c) cooling the boiled wort.

Step (a):

The malt powder can be obtained by germinating barley, for example, two-rowed barley, by a routine method and drying this barley, followed by pulverization to the predetermined particle size. Examples of the secondary raw materials include rice, corn starch, corn grits and sugars. The quality of water may influence the texture or flavor/taste of the resulting product in some cases and, however, is not particularly limited as long as the effect of the present invention is exerted. In this context, the proportions of the malt powder, the secondary raw materials and water in the mixture can be determined appropriately. Also, the saccharification and filtration of such a mixture can be performed according to a routine method.

Step (b):

It is preferred that hops should be added to the wort thus obtained, followed by boiling. This boiling can bring out the flavor and aroma of the hops. It is also preferred that precipitates such as proteins should be removed after the boiling.

Step (c):

Subsequently, the boiled wort is cooled. For this cooling, it is desirable that the wort should be cooled to a temperature as low as possible at which the wort is not frozen, usually, 1 to 5° C.

### (Reduction of Off-Flavor)

In the present invention, the alcohol-free malt beverage to which health functions of ornithine is imparted is prepared as a malt beverage containing substantially no alcohol by a preparation process free from a fermentation step. In this context, unfermented wort prepared in the production of fermented malt beverages such as beer usually has a specific strong off-flavor (odor of wort) resulting in a flavor far from beer. It is known that even slight fermentation produces such a strong off-flavor. Thus, hops or a processed product derived from hops, for example, a fraction of hop essential oil components is used to reduce this off-flavor and adjust it to a more preferable flavor. In addition, the content of a hop-derived aroma component myrcene, α-humulene or β-selinene in the malt beverage is adjusted to a particular value. The malt beverage thus produced can have a reduced off-flavor, retain the aroma components of hops and have no alcohol content.

The adjustment of the hop-derived aroma component in the malt beverage is performed by the timing of addition of hops and/or hop essential oil components in the wort boiling step, the amount of the hops and/or hop essential oil components added, or the amount of the hop essential oil components added after the wort boiling step. For example, the addition of 2 g/L hops during 90-minute boiling can effectively reduce the off-flavor. Similar effect can also be obtained by the addition of hop essential oil in a post-boiling step without adding hops during boiling. However, the addition of hops in a larger amount at a timing closer to the completion of boiling or the addition of hop essential oil in a larger amount in a post-boiling step strengthens the resin-like odor of the hops and rather imparts the off-flavor thereto, resulting in a far out-of-balance flavor.

In the production of the malt beverage having no alcohol content according to the present invention, it is preferred that the content of the hop-derived aroma component myrcene, a-humulene or β-selinene in the malt beverage should be adjusted so that: a marker compound value determined by GC/MS (GC mass spectrum) for obtaining off-flavor (odor of wort)-reducing effect is 2.5 ppb or more myrcene, 5.2 ppb or more a-humulene, or 1.5% or more β-selinene as a value quantified in terms of a response ratio of 93 m/z quantitative ion to 110 m/z ion of an internal standard borneol; and a marker compound value for preventing hop-derived unpleasant smell is less than 187.5 ppb myrcene or less than 65.1 ppb a-humulene. Such adjustment of the hop-derived aroma component in the malt beverage can reduce the off-flavor in a manner independent of fermentation and achieves the preparation of an alcohol-free malt beverage retaining the aroma components of hops.

Step (d):

The wort thus obtained may be treated with an adsorbent for removing the wort flavor to remove the wort flavor by adsorption.

### (Filtration of Malt Beverage)

The malt beverage thus obtained is subsequently filtered to remove unnecessary proteins and the adsorbent. The filtration can be performed using a diatomaceous earth filter. In the present invention, for example, steps performed in typical beer or sparkling liquor production can be performed, for example, adjustment of the final concentration using degassed water or the like, low-temperature sterilization (pasteurization), charging into containers (e.g., barrels, bottles, or cans) (packaging), and labeling of the containers.

### (pH Adjustment)

The pH of the product must be decreased to the predetermined value based on the food sanitation law. The pH adjuster can be added in any stage during the production method described above. Examples of an organic acid added for this pH adjustment include one or more organic acids selected from lactic acid, malic acid, gluconic acid and phytic acid. It is preferred that the organic acid should be added to adjust the pH value of the product to lower than 3.80.

### (Addition of Ornithine)

In the present invention, ornithine that can be used to impart its health function to an alcohol-free malt beverage is ornithine hydrochloride and ornithine aspartate usually used as ornithine salts. In the present invention, it is preferred for imparting health functions of ornithine to an alcohol-free malt beverage that ornithine hydrochloride and ornithine aspartate are contained in an amount of at least 0.05% or more by weight in terms of ornithine per product of the alcohol-free malt beverage.

In the present invention, it is preferred that the content of ornithine per product of the alcohol-free malt beverage should be set to 0.05% by weight to 0.2% by weight. For canned beverages, the amount of ornithine is preferably 0.2% by weight, more preferably 0.17% by weight, as the upper limit from the viewpoint of the corrosiveness of the cans and umami. In the present invention, it is also preferred that ornithine aspartate should be contained in an amount of 500 ppm or lower. For effectively exerting the health functions of ornithine in commercial use of the alcohol-free malt beverage of the present invention provided with the health functions of ornithine, it is preferred that the ornithine content per beverage should be adjusted to the range of 250 mg to 2000 mg, more preferably the range of 250 mg to 1000 mg.

In the present invention, ornithine hydrochloride and ornithine aspartate to be added as ornithine raw materials are used in combination at such a weight ratio that 20% or more by weight of ornithine hydrochloride in terms of ornithine is used with respect to the total weight of ornithine hydrochloride and ornithine aspartate in terms of ornithine. The suitable weight ratio between ornithine hydrochloride and ornithine aspartate used can be determined appropriately according to the type of the organic acid used for pH adjustment and the amount of the organic acid added.

For example, in the case where malic acid is used as the organic acid, when the amount of the malic acid added is 0.058 to 0.078% v/v with respect to wort, 80% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=80:20.

Furthermore, when the amount of the gluconic acid added is 0.145 to 0.24% v/v with respect to wort, 60% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=60:40 to 80:20, more preferably 60:40. Alternatively, when the amount of the phytic acid added is 0.03 to 0.06% v/v with respect to wort, 20% or more by weight of ornithine hydrochloride in terms of ornithine is used with respect to the total weight of ornithine hydrochloride and ornithine aspartate in terms of ornithine. Preferably, the weight ratio between the amounts of ornithine hydrochloride and ornithine hydrochloride used is ornithine hydrochloride:ornithine aspartate=20:80 to 80:20, more preferably 20:80 to 60:40.

The alcohol-free malt beverage provided with the health functions of ornithine is produced by the method described above. Ornithine can be added in any stage during the production process of the alcohol-free malt beverage. Materials containing ornithine hydrochloride and ornithine aspartate for adding ornithine are not particularly limited. Possible materials are, for example, ornithine hydrochloride and ornithine aspartate themselves or free forms of these ornithines (solution having 100% ornithine content).

Hereinafter, the present invention will be described specifically with reference to Examples. However, the present invention is not intended to be limited to Examples below.

### Example 1

### (1) Preparation of Wort

730 L of hot water was added and mixed into 240 kg of malt grist in a mash tun and kept at 50 to 70° C. for 10 to 90 minutes to prepare Maische. This Maische was subsequently kept at 68 to 76° C. for 10 to 40 minutes for saccharification. After the completion of the saccharification step, the resulting product was filtered in a wort filtration tank to obtain 2,000 L of transparent wort as the filtrate (sugar content: 5.0%). The obtained wort was transferred to a boiling kettle. Further, 1.1 kg of hops were added thereto, and the mixture was boiled at 100° C. for 90 minutes. To the boiled wort, ornithine hydrochloride and ornithine aspartate were added at ratios of 6 levels shown in Table 1. Finally, cooled wort having an ornithine concentration adjusted to 990 to 1300 mg/L was prepared. To the cooled wort, lactic acid, malic acid, gluconic acid or phytic acid was added to decrease the pH value to 3.80. Finally, the ornithine concentration was adjusted to 990 to 1300 mg/L.

Test section

| | 1 | 2 | 3 | 4 | 5 | 6 |
|---|---|---|---|---|---|---|
| Ratio of ornithine | 0 | 20 | 40 | 60 | 80 | 100 |
| hydrochloride | | | | | | |
| Ratio of ornithine | 100 | 80 | 60 | 40 | 20 | 0 |
| aspartate | | | | | | |

*The ratios of ornithine hydrochloride and ornithine aspartate are indicated in terms of ornithine concentration.

### (2) Quality

As a result of sensory evaluation of the obtained wort, the higher percentage of ornithine hydrochloride achieved a lower amount of the organic acid used resulting in the lower intensity of sourness. Also, the intensity of sourness differed depending on the type of the organic acid. Use of phytic acid or gluconic acid achieved the reduced intensity of sourness. The results are shown in Table 2.

| | | Test section | | | | | |
|---|---|---|---|---|---|---|---|
| | | 1 | 2 | 3 | 4 | 5 | 6 |
| Ratio of hydrochloride in | | 0 | 20 | 40 | 60 | 80 | 100 |
| terms of ornithine | | | | | | | |
| Ratio of aspartate in | | 100 | 80 | 60 | 40 | 20 | 0 |
| terms of ornithine | | | | | | | |
| Amount | Lactic acid | 0.162 | 0.135 | 0.117 | 0.09 | 0.0855 | 0.0693 |
| (% v/v per | Malic acid | 0.138 | 0.12 | 0.106 | 0.092 | 0.078 | 0.058 |
| 100%) of | Gluconic | 0.33 | 0.3 | 0.27 | 0.24 | 0.19 | 0.145 |
| organic acid | acid | | | | | | |
| used required | Phytic acid | 0.065 | 0.06 | 0.055 | 0.0475 | 0.04 | 0.03 |
| for pH | | | | | | | |
| adjustment to | | | | | | | |
| 3.80 | | | | | | | |
| Intensity of | Lactic acid | 1 | 1.2 | 1.8 | 2.6 | 3 | 3.4 |
| sourness | (90%) | | | | | | |
| evaluated on | Malic acid | 1 | 1.4 | 2.2 | 2.6 | 3.2 | 3.8 |
| scale of 1 to 5 | (20%) | | | | | | |
| *1: strongly | Gluconic | 1.6 | 2 | 2.8 | 3.4 | 3.6 | 4.6 |
| sour | acid (50%) | | | | | | |
| | Phytic acid | 2.6 | 3.2 | 3.8 | 4 | 4.4 | 5 |
| | (50%) | | | | | | |
| Suitability | Lactic acid | 1 | 1.2 | 1.8 | 2.2 | 2.8 | 3.2 |
| as beverage | (90%) | | | | | | |
| evaluated on | Malic acid | 1 | 1.2 | 1.8 | 2.4 | 3 | 3.4 |
| scale of 1 to 5 | (20%) | | | | | | |
| *3 or higher: | Gluconic | 1.4 | 1.8 | 2.6 | 3.2 | 3.6 | 4.4 |
| suitable as | acid (50%) | | | | | | |
| beverage | Phytic acid | 2.4 | 3.2 | 3.4 | 4 | 4.2 | 5 |
| | (50%) | | | | | | |
| Intensity of | Evaluation | 1 | 1.8 | 2.6 | 3 | 4 | 5 |
| umami | using only | | | | | | |
| evaluated on | phytic acid | | | | | | |
| scale of 1 to 5 | (50%) | | | | | | |
| *1: | | | | | | | |
| aftertaste | | | | | | | |
| derived from | | | | | | | |
| umami was | | | | | | | |
| present | | | | | | | |

| | | | | | | |
|---|---|---|---|---|---|---|
| Aspartate (mg/L) | 1242.0 | 957.7 | 810.1 | 502.5 | 298.7 | 26.5 |
| Chlorine concentration | 0.0 | 64.9 | 129.9 | 194.8 | 259.7 | 324.7 |
| (calculated value) (mg/L) | | | | | | |
| Predicted chloride | 80.0 | 144.9 | 209.9 | 274.8 | 339.7 | 404.7 |
| concentration: plus 80 mg/L | | | | | | |
| derived from wort | | | | | | |

In the evaluation shown above, [Intensity of sourness] was evaluated on a scale of 1 to 5 wherein score represents being strongly sour. [Suitability as beverage] was evaluated on a scale of 1 to 5 wherein score or higher represents being suitable as a beverage. [Intensity of umami] was evaluated on a scale of 1 to 5 wherein score 1 represents that aftertaste derived from umami was present whereas score 5 represents that the beverage had clear, refreshing taste with little aftertaste derived from umami. [Chlorine concentration] represents a calculated value of a chlorine concentration derived from ornithine hydrochloride. Ornithine hydrochloride has an ornithine:chloride ion ratio of 80:20%. Since wort-derived chloride ion was also present, a predicted value of the total chlorine concentration is indicated by “Predicted chloride concentration: plus 80 mg/L derived from wort”. A chlorine concentration of 200 ppm or higher usually increases the risk of the corroding cans and therefore requires testing the presence or absence of such corrosiveness beforehand. In this regard, the reliable chlorine concentration is 300 ppm or lower in consideration of past performance, though this is not general findings.

### Evaluation>

The test shown above demonstrated that the followings:

(a) The higher percentage of ornithine hydrochloride achieved the lower intensity of sourness as a whole.

This is because ornithine hydrochloride does not increase the buffering capacity of wort and as such, achieved pH 3.8 with a small amount of an organic acid. It should be understood that ornithine hydrochloride:ornithine aspartate=20:80 to 100:0 is appropriate from the viewpoint of sourness (however, depending on the type of the organic acid used).

(b) The higher percentage of ornithine hydrochloride rather leads to the higher concentration of chloride ions. In general, the increased chlorine concentration is responsible for the corrosiveness of production equipment or can materials. It should be understood that ornithine hydrochloride:ornithine aspartate=0:100 to 80:20, more preferably, ornithine hydrochloride:ornithine aspartate=0:100 to 60:40 is appropriate from the viewpoint of chloride ion concentrations.

(c) The intensity of sourness was found to correlate with suitability as a beverage. Since the organic acid was used in a large amount to achieve pH less than 3.80, many test sections exhibited strong sourness. Stronger sourness resulted in lower suitability as a beverage. It should be understood that ornithine hydrochloride:ornithine aspartate=20:80 to 100:0 is appropriate from the viewpoint of suitability as a beverage (however, depending on the type of the organic acid used).

(d) The lower percentage of ornithine aspartate achieved the lower intensity of umami. Excessive umami lingers as aftertaste and deletes refreshment. The aspartic acid concentration adjusted to 500 ppm or lower successfully prevented the unpleasant aftertaste of umami. It should be understood that ornithine hydrochloride:ornithine aspartate=40:60 to 100:0 is appropriate from the viewpoint of the reduction of unpleasant aftertaste of umami.

In conclusion, it was demonstrated that the production method of the present invention is suitable for the preparation of an ornithine-containing alcohol-free malt beverage which has sourness balanced with umami and is excellent in flavor. It was also demonstrated that ornithine hydrochloride:ornithine aspartate=20:80 to 100:0 (provided that ornithine hydrochloride:ornithine aspartate=20:80 is for phytic acid used) is suitable for the preparation of an ornithine-containing alcohol-free malt beverage from the viewpoint of suitability as a beverage.

It was concluded that ornithine hydrochloride:ornithine aspartate=20:80 to 80:20, more preferably ornithine hydrochloride:ornithine aspartate=40:60 to 80:20, further preferably ornithine hydrochloride:ornithine aspartate=40:60 to 60:40 is appropriate from the overall viewpoint including suitability as a beverage as well as the aftertaste of umami and the lowering of chloride ion concentrations. Phytic acid or gluconic acid was confirmed to be particularly preferable as the organic acid.

### INDUSTRIAL APPLICABILITY

The present invention provides an ornithine-containing alcohol-free malt beverage which is provided with health functions of ornithine imparted to an alcoholic beverage-flavored alcohol-free malt beverage containing substantially no alcohol component and which retains an excellent flavor of the alcohol-free malt beverage.

1. A method for producing an ornithine-containing alcohol-free malt beverage which is provided with health functions of ornithine and retains an excellent flavor of an alcohol-free malt beverage, comprising using ornithine hydrochloride and ornithine aspartate in combination as ornithine raw materials to produce the ornithine-containing alcohol-free malt beverage, wherein 20% or more by weight of ornithine hydrochloride in terms of ornithine is used with respect to the total weight of ornithine hydrochloride and ornithine aspartate in terms of ornithine.

2. The method for producing an ornithine-containing alcohol-free malt beverage which is provided with health functions of ornithine and retains an excellent flavor of an alcohol-free malt beverage according to claim 1, wherein 60% or more by weight of ornithine hydrochloride in terms of ornithine is used with respect to the total weight of ornithine hydrochloride and ornithine aspartate in terms of ornithine.

3. The method for producing an ornithine-containing alcohol-free malt beverage which is provided with health functions of ornithine and retains an excellent flavor of an alcohol-free malt beverage according to claim 2, wherein 80% or more by weight of ornithine hydrochloride in terms of ornithine is used with respect to the total weight of ornithine hydrochloride and ornithine aspartate in terms of ornithine.

4. The method for producing an ornithine-containing alcohol-free malt beverage which is provided with health functions of ornithine and retains an excellent flavor of an alcohol-free malt beverage according to claim 1, wherein ornithine hydrochloride and ornithine aspartate are contained in an amount of at least 0.05% or more by weight in terms of ornithine per product of the alcohol-free malt beverage.

5. The method for producing an ornithine-containing alcohol-free malt beverage which is provided with health functions of ornithine and retains an excellent flavor of an alcohol-free malt beverage according to claim 1, wherein ornithine aspartate is contained in an amount of 500 ppm or lower.

6. The method for producing an ornithine-containing alcohol-free malt beverage which is provided with health functions of ornithine and retains an excellent flavor of an alcohol-free malt beverage according to claim 1, wherein the method further comprises adding an organic acid to adjust the pH value of the product, in addition to using ornithine hydrochloride and ornithine aspartate in combination as ornithine raw materials, to produce the ornithine-containing alcohol-free malt beverage.

7. The method for producing an ornithine-containing alcohol-free malt beverage which is provided with health functions of ornithine and retains an excellent flavor of an alcohol-free malt beverage according to claim 6, wherein the added organic acid is one or more organic acids selected from malic acid, gluconic acid and phytic acid.

8. The method for producing an ornithine-containing alcohol-free malt beverage which is provided with health functions of ornithine and retains an excellent flavor of an alcohol-free malt beverage according to claim 6, wherein the organic acid is added to adjust the pH value of the product to less than 3.80.

9. The method for producing an ornithine-containing alcohol-free malt beverage which is provided with health functions of ornithine and retains an excellent flavor of an alcohol-free malt beverage according to claim 7, wherein malic acid is used in an amount of 0.058 to 0.078% v/v with respect to wort, and 20% or more by weight of ornithine hydrochloride in terms of ornithine is used with respect to the total weight of ornithine hydrochloride and ornithine aspartate in terms of ornithine.

10. The method for producing an ornithine-containing alcohol-free malt beverage which is provided with health functions of ornithine and retains an excellent flavor of an alcohol-free malt beverage according to claim 7, wherein gluconic acid is used in an amount of 0.145 to 0.24% v/v with respect to wort, and 40% or more by weight of ornithine hydrochloride in terms of ornithine is used with respect to the total weight of ornithine hydrochloride and ornithine aspartate in terms of ornithine.

11. The method for producing an ornithine-containing alcohol-free malt beverage which is provided with health functions of ornithine and retains an excellent flavor of an alcohol-free malt beverage according to claim 7, wherein phytic acid is used in an amount of 0.03 to 0.06% v/v with respect to wort, and 80% or more by weight of ornithine hydrochloride in terms of ornithine is used with respect to the total weight of ornithine hydrochloride and ornithine aspartate in terms of ornithine.

12. The method for producing an ornithine-containing alcohol-free malt beverage which is provided with health functions of ornithine and retains an excellent flavor of an alcohol-free malt beverage according to claim 1, wherein the alcohol-free malt beverage is produced by a nonfermentation process of wort.

13. An ornithine-containing alcohol-free malt beverage which is provided with health functions of ornithine and retains an excellent flavor of an alcohol-free malt beverage, produced by a method for producing an alcohol-free malt beverage according to claim 1.

14. An ornithine-containing alcohol-free malt beverage which is provided with health functions of ornithine and retains an excellent flavor of an alcohol-free malt beverage, comprising 0.05% or more by weight and 0.2% or less by weight of ornithine.
